(19) United States
(12) Patent Application Publication
(10) Pub. No.: US 2019/0032116 A1
(43) Pub. Date: Jan. 31, 2019

(54) SEQUENCE SPECIFIC METHYLATION ENRICHMENT AND DETECTION

(71) Applicant: GENETICS RESEARCH, LLC, D/B/A ZS GENETICS, INC., Wakefield, MA (US)

(72) Inventor: Anthony P. Shuber, Wakefield, MA (US)

(21) Appl. No.: 16/150,962

(22) Filed: Oct. 3, 2018

Related U.S. Application Data
(63) Continuation-in-part of application No. 16/018,926, filed on Jun. 26, 2018.
(60) Provisional application No. 62/672,217, filed on May 16, 2018.
(60) Provisional application No. 62/526,091, filed on Jun. 28, 2017.

(51) Int. Cl. C12Q 1/6816; C12Q 1/6809; C12N 9/88; C12N 15/10

(52) U.S. Cl. CPC C12Q 1/6816; C12Q 1/6809; C12N 2310/20; C12N 15/1013; C12Q 2600/154; C12N 9/88

(57) ABSTRACT

The invention provides methods for detecting epigenetic changes, including but not limited to methylation changes, directly from biological samples, without the need for certain complex sample preparation steps. The invention provides Cas protein/guide RNA complexes that may be introduced directly into the sample, where the complexes target and bind the target region. The target region is thus enriched and isolated in a sequence-specific manner. The target region may then be subject to any suitable signal amplification assay to detect the epigenetic change in the target region. Detection of DNA hypermethylation in the target region is indicative of disease, such as cancer.

### CROSS REFERENCE TO RELATED APPLICATIONS

This application is a continuation-in-part of U.S. Non-Provisional application Ser. No. 16/018,926, filed Jun. 26, 2018, which claims priority to and the benefit of U.S. Provisional Application No. 62/526,091, filed Jun. 28, 2017 and U.S. Provisional Application No. 62/672,217, filed May 16, 2018, the contents of each of which are incorporated by reference.

### TECHNICAL FIELD

The invention relates to molecular genetics.

### BACKGROUND

When testing for diseases, such as cancer, physicians often rely on liquid and tissue biopsy. Conventional biopsy sample analysis methods typically require expensive and time-consuming sample preparation procedures, kits, and reagents.

For example, in a liquid biopsy, a blood sample is obtained from a patient and may be centrifuged to remove whole blood cells, leaving plasma or serum that includes cell-free DNA (cfDNA). Typically, the sample must be subject to a sample preparation protocol before any genetic analysis is performed. For example, laboratory technicians use a commercially-available kit to aliquot the serum through a series of steps that use proteinase solutions to digest away proteins, lysis buffers to dissociate vesicles and other lipid fragments, and cleaning and suspension buffers. In some protocols, the resultant mixture is washed through a membrane within a column under vacuum after which the cfDNA is eluted from the column with a specialty wash buffer. The entire process can require hours or more and the use of expensive kits.

Epigenetic changes, such as DNA methylation, are common in the genome and can be indicative of disease. Methylation is the most studied epigenetic change and has been linked to cancer and certain metabolic disorders. Methylation is a chemical modification of DNA in which a methyl group is added to certain cytosines in DNA to yield 5-methylcytosine, Methylation appears to influence gene expression by affecting the interactions of both chromatin proteins and specific transcription factors with DNA. In cancer cells, hypermethylation or hypomethylation of a specific region of DNA is associated with cancer. The detection of, for example, hypermethylation in regions associated with cancer provides a useful approach to early detection.

Conventional protocols for detecting methylation utilize bisulfite treatment to convert unmethylated cytosine to uracil in the DNA. Any methylated cytosine will not be converted in response to bisulfite treatment. Then, various sequencing techniques such as bisulfite sequencing or methylation-specific PCR, are used to detect methylated cytosines. These methods are not only time consuming and require expensive kits, but damage to the DNA and incomplete conversion during bisulfite treatment, render them unsuitable for reliable and targeted sequence-specific analysis.

Methylation is not the only epigenetic change that has been associated with disease. Histone modification, chromatin rearrangement, and RNA silencing are examples of other epigenetic modifications that can have an impact on health and disease. Since epigenetic changes may be good diagnostic markers for disease, there remains a need for rapid and simple targeted detection.

### SUMMARY

The present invention provides methods for detecting epigenetic changes, including but not limited to methylation changes, in DNA directly from biological samples without the need for significant sample preparation steps or kits.

Methods of the invention use Cas endonuclease to bind target regions of DNA suspected to be affected by epigenetic changes. According to the invention, Cas endonuclease is complexed with one or more guide RNAs that bind to protospacer adjacent motif (PAM) sequences near a region of interest, such as a methylation locus known to be associated with cancer, when hypermethylated. The Cas endonuclease binds to and protects target regions of DNA even when a target DNA is only present as a small fraction of the sample. Thus, methods of the invention are useful when analyzing DNA present in low abundance in a sample such as blood or other bodily fluids.

In a preferred embodiment, Cas proteins, along with their sequence-specific guide RNAs (gRNA), are complexed and introduced directly into the biological sample. The Cas/guide RNA complex may be introduced as part of sample collection, or added into collection tubes containing the sample. The gRNA mediates binding of the Cas proteins to a target region of DNA of interest, such as a tumor DNA fragment suspected to be hypermethylated.

The target region is enriched relative to other materials in the sample by any suitable enrichment methods, such as by elution of bound Cas proteins. The target region may be enriched by elimination of non-target DNA acid using, for example, exonucleases. Enrichment methods may be used alone or in combination with other enrichment methods. As a non-limiting example, the sample is enriched by isolating complex-bound target regions by amplification, size fractionation, or hybrid capture. In another embodiment, exonuclease digestion may be used alone, or may be used before or after elution of bound Cas proteins.

Epigenetic modifications are detected using available methods. For example, for methylation, one can use antibody binding, immunoassay, amplification, or gel electrophoresis, or other techniques known in the art. Detection of DNA methylation in the target region is indicative of increased risk, or even presence, of cancer.

Methods and related kits described herein are useful to detect the presence of methylation in a sample. Due to the nature by which a protein, such as a Cas complex, binds nucleic acid, methods may be used even where the target is present only in very small quantities, e.g., even as low as 0.01% frequency of mutant fragments among normal fragments in a sample (i.e., where about 50 copies of a circulating tumor DNA fragment harboring hypermethylation sites are present among about 500,000 unrelated fragments of similar size). Thus, methods of the invention may have particular applicability in discovering very rare, yet clinically important, information, such as site-specific methylation of tumor related genes and may be used to detect abnormal methylation within cell-free DNA, such as target region-specific hypermethylation in circulating tumor DNA.

In a preferred method, CRISPR/Cas systems and associated guide RNAs are introduced to a biological sample. When used according to methods of the invention, Cas endonuclease—whether catalytically active or inactive—will bind to a target consistently via a guide RNA and will protect that target (i.e., stay bound), thereby allowing the target to be pulled out of the sample by, for example, elution of the captured sequence or elimination of non-target DNA. In certain aspects, the invention provides methods for detecting hypermethylation of a target region of DNA. Methods include obtaining a biological sample from a subject, introducing Cas proteins and guide RNA into the sample, and binding the Cas proteins to ends of a target region of DNA. The Cas protein may be a Cas endonuclease or a catalytically deficient homolog thereof. The target region is then enriched by isolating the target region from the sample.

The target region may be any naturally-occurring or artificial DNA. In other embodiments, the nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target region or feature of interest may be any site of epigenetic modification that is indicative of disease, such as a nucleic acid modification, a histone modification, or chromatin remodeling. An example of a nucleic acid modification may be a modification to methylation status of DNA. As such, a feature of interest may be DNA hypermethylation or hypomethylation. In certain embodiments, the methods of the invention involve the detection of DNA hypermethylation of the target region.

The target may be from a sub-population of nucleic acid within the nucleic acid sample. For example, the target may contain cell-free DNA, such as cell-free fetal DNA or circulating tumor DNA. In some embodiments, the sample includes plasma from the subject and the target is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target is complementary DNA (cDNA), which is made by reverse transcribing RNA. In some embodiments, detecting cDNA is a way to detecting target RNA.

The target may be from any source of DNA. In preferred embodiments, the target is DNA from a biological sample from a human or other animal. In preferred embodiments, the biological sample is a body fluid sample, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the biological sample is a blood sample, serum sample, plasma sample, urine sample, saliva sample, semen sample, feces sample, phlegm sample, or liquid biopsy. The biological sample may be a tissue sample from an animal, such as skin, conjunctiva, gastrointestinal tract, respiratory tract, vagina, placenta, uterus, oral cavity or nasal cavity. The biological sample may be a liquid biopsy or a tissue biopsy.

In some embodiments, obtaining the sample includes obtaining a biological sample from a subject in a collection tube. In a non-limiting example, the biological sample is blood and the collection tube is centrifuged to isolate serum or plasma from blood cells. The Cas endonuclease or catalytically deficient homolog thereof is introduced into the serum or plasma. In a preferred embodiment, the Cas endonuclease, or the catalytically deficient homolog thereof, is complexed with the guide RNA and introduced into the serum or plasma. In an embodiment, the Cas endonuclease, or the catalytically deficient homolog thereof, is introduced into the serum or plasma as a ribonucleoprotein (RNP) in which the endonuclease is complexed with the guide RNA. Preferably, the guide RNA includes at least two single guide RNA molecules that each complex with a Cas endonuclease, where the complexes target PAM sequences that are adjacent a target region and the guide RNA guides Cas endonuclease to hybridize to one of the targets, wherein the target region includes a methylation site associated with cancer when hypermethylated.

The method may include enriching the sample by isolating the complex-bound target region from some or all of the unbound DNA. For example, the method may include binding the complex-bound target region to a particle. The particle may include magnetic or paramagnetic material. The method may include applying a magnetic field to the sample. The particle may include an agent that binds to a protein bound to an end of the target DNA. The agent may an antibody or fragment thereof. The method may include chromatography, applying the sample to a column, or gel electrophoresis. The method may include separating the complex-bound target from some or all of the unbound nucleic by size exclusion, ion exchange, or adsorption.

Each of the proteins may independently be any protein that binds a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a catalytically inactive form of a nuclease, such as a programmable nuclease described above. The protein may be a transcription activator-like effector (TALE). The protein may be complexed with a nucleic acid that guides the protein to an end of the nucleic acid. For example, the protein may be a Cas endonuclease in a complex with one or more guide RNAs. Preferably, the protein is a Cas endonuclease or a catalytically deficient homolog thereof. In a preferred embodiment, the Cas endonuclease is in a complex with one or more guide RNAs.

Once the sample is enriched, the target region may be detected by any means known in the art. For example and without limitation, the target region may be detected by ligand binding assay, DNA staining, spectrophotometry, sequencing, fluorescent probe hybridization, fluorescence resonance energy transfer, optical microscopy, or electron microscopy. Methods of the invention may include detecting and quantifying the DNA methylation of the target region. Detecting and quantifying the DNA methylation of the target region may include ligand binding assays, immunosorbent assays, such as enzyme-linked immunosorbent assay (ELISA), or other signal amplification techniques. In certain embodiments, the methods include quantifying relative amounts of hypermethylation of the target region.

In some embodiments, the target is DNA and includes hypermethylation specific to a tumor. In some other embodiments, the target is nucleic acid and includes a mutation specific to a tumor. The target may be present at no more than about 0.01% of cell-free DNA in the plasma or serum. By methods herein, the target is enriched by isolating the target from the serum or plasma.

Furthermore, methods of the invention may include negative enrichment. As an example, Cas endonuclease may be provided with one or more guide RNAs that targets and binds to one or more target regions of DNA, such as a region or locus suspected to be hypermethylated. In other embodiments, the Cas endonuclease/guide RNA complex targets and binds to a target nucleic acid and flanks a loci of interest, such as a locus of a known cancer-associated mutation or a specific genetic allele of clinical interest. The Cas endonuclease binds to, and protects, DNA containing hypermethylated sites even when the mutation is only present as a small fraction of the sample. In other embodiments, the Cas endonuclease binds to, and protects mutation-containing nucleic acid even when the mutation is only present as a small fraction of the sample. The bound Cas proteins prevent exonuclease from digesting the target and, after incubation with exonuclease, the only nucleic acid substantially present in the sample is that of the target. The target is thus isolated or enriched in a sequence-specific manner. The target may then be subject to any suitable detection or analysis assay such as antibody signal or immunosorbent assays.

In a preferred method, CRISPR/Cas systems using guide RNAs specific for a methylation site is introduced to the sample under conditions such that DNA containing the methylation site is protected from exonuclease digestion while non-target DNA is digested by an exonuclease. When used according to methods of the invention, Cas endonuclease—whether catalytically active or inactive—will bind to a target consistently via a guide RNA and will protect that target (i.e., stay bound) for at least long enough that a promiscuous exonuclease can be reliably used to digest unbound, non-target DNA. By protection of the target with digestion of the non-target, a sample is effectively enriched for the target, and those remaining target fragments are captured, stored, isolated, preserved, detected, sequenced, or otherwise assayed with success that would be unobtainable without methods of the invention.

In other embodiments of the invention, CRISPR/Cas systems using guide RNAs specific for a mutation is introduced to the sample under conditions such that nucleic acid containing the mutation is protected from exonuclease digestion while non-target nucleic acid is digested by an exonuclease. When used according to methods of the invention, Cas endonuclease—whether catalytically active or inactive—will bind to a target consistently via a guide RNA and will protect that target (i.e., stay bound) for at least long enough that a promiscuous exonuclease can be reliably used to digest unbound, non-target nucleic acid. By protection of the target with digestion of the non-target, a sample is effectively enriched for the target, and those remaining target fragments are captured, stored, isolated, preserved, detected, sequenced, or otherwise assayed with success that would be unobtainable without methods of the invention. In certain aspects, the invention provides a method for detecting a target nucleic acid. The method includes obtaining a serum or plasma sample from a subject, introducing Cas proteins and guide RNA into the serum or plasma, and binding the Cas proteins to ends of a target nucleic acid. The Cas protein may be a Cas endonuclease or a catalytically deficient homolog thereof. Unbound nucleic acid is digested from the sample by introducing exonuclease while the Cas proteins prevent the exonuclease from digesting the target nucleic acid, thereby enriching the sample for the target nucleic acid. The target nucleic acid may then be isolated from the enriched sample by amplification, size fractionation, or hybrid capture. Methods may include inactivating the exonuclease (e.g., by heating) prior to the isolating step. Preferably, two Cas proteins bind to ends of the target nucleic acid and prevent the exonuclease from digesting the target nucleic acid.

In a preferred embodiment, the invention provides a method for detecting methylation in DNA. The method includes obtaining a biological sample, exposing the biological sample to a Cas/guide RNA complex. The Cas protein may be a Cas endonuclease or a catalytically deficient homolog thereof. The complex targets and binds to one or more target regions of DNA suspected to be hypermethylated in the sample. Preferably, the complexes are targeted to PAM sequences that are adjacent a target region of DNA suspected to be hypermethylated in the sample. The sample may be enriched by isolating the target DNA by amplification, size fractionation, and hybrid capture.

In other embodiments, the sample may be enriched by digesting the unbound DNA by introducing exonuclease while the Cas proteins prevent the exonuclease from digesting the target region, thereby enriching the sample for the target region DNA. As such, methods may include inactivating the exonuclease (e.g., by heating) prior to isolating the target DNA.

### BRIEF DESCRIPTION OF THE DRAWINGS

FIG. 1 shows a table of the inputs and the dilation amounts used in the Example described herein. Dilution 11 is at 3× concentration from previous experiments because the experiment uses 3× as much input DNA volume in the reaction. The copies per ul of stock, copies per ul in 50 ul reaction, amount of previous dilution (ul), plasma, and total volume (ul) are indicated.

FIG. 2 shows a table of the dilutions used in the Example. For the percent of plasma in the final reaction, the percent of plasma in 2× sample, plasma dilution (ul), and tris dilution (ul) are shown in the table.

FIG. 3 shows a graph of the qPCR results after amplification from the post-cutting dilutions described in the Example.

FIG. 4 shows the tabulated qPCR results from the Example. Percent plasma, use of a Streck tube, amount of no Cas9 present, amount of Cas9 present, and percent cutting are indicated.

FIG. 5 shows a chart of the binding efficiency from the Example, particularly showing the relationship between percent cleavage and percent plasma. In particular, the percent cleavage is shown as a function of the amount or percent of plasma in the cutting reaction. Results are shown for samples with no tube and samples using a Streck tube.

FIG. 6 shows a chart of the detection signal in plasma from the Example, particularly showing the relationship between qPCR signal and percent plasma. In particular, the percent detection of no plasma in the sample is shown as a function of the percent plasma in the cutting reaction. Results are shown for samples with no tube and sample using a Streck tube.

### DETAILED DESCRIPTION

Methods of the invention provide for the enrichment of a target nucleic acid, in a sequence-specific manner, directly from bodily fluid samples without the need for complex sample preparation. Preferred embodiments include obtaining a bodily fluid sample from a subject. Certain embodiments of the invention provide a method for detecting a target nucleic acid in the bodily fluid sample. Certain other embodiments of the invention also provide for the detection of methylation in DNA, in a sequence-specific manner, directly from biological samples without the need for complex sample preparation or sequencing. The sample is positively enriched for the target region and methylation is detected. In preferred embodiments of the invention, the target region is suspected to be hypermethylated.

Methods of the invention include introducing the Cas endonuclease, catalytically inactive Cas endonuclease, or homolog thereof and guide RNA into the bodily fluid sample. In a preferred embodiment, the binding proteins are provided by Cas endonuclease/guide RNA complexes. Embodiments of the invention use Cas endonuclease proteins that are originally encoded by genes that are associated with clustered regularly interspaced short palindromic repeats (CRISPR) in bacterial genomes. A CRISPR-associated (Cas) endonuclease may be introduced directly into the bodily fluid sample.

The Cas proteins bind to ends of a target nucleic acid. The target nucleic acid is thus isolated or enriched in a sequence-specific manner. The enriched target nucleic acid may then be subject to any suitable detection or analysis assay such as amplification or sequencing. The enriched target nucleic acid may be further enriched by digesting other, unbound nucleic acids present in the sample with exonuclease. The bound Cas proteins prevent the exonuclease from digesting the target nucleic acid, thereby leaving the only the target nucleic acid substantially present in the sample. The target nucleic acid is thus isolated or enriched in a sequence-specific manner. The target nucleic acid may then be subject to any suitable detection or analysis assay such as amplification or sequencing. The target may be subjected to a signal amplification assay, for example, an immunoassay.

Preferably, the Cas endonuclease is complexed with a guide RNA that targets the Cas endonuclease to a specific sequence. In a preferred embodiment, the Cas endonuclease/guide RNA complex targets and binds to one or more target regions of DNA suspected to be hypermethylated. Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease and a first guide RNA. In the depicted embodiment, the complex comprises the Cas endonuclease or the catalytically deficient homolog thereof being introduced into the serum or plasma as a ribonucleoprotein (RNP) in which the Cas endonuclease or catalytically deficient homolog thereof is complexed with the guide RNA. The Cas endonuclease will bind to the target. The target may then be isolated or enriched, allowing for detection of the target.

The proteins that bind to ends of the target nucleic acid may be any proteins that bind to a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

The protein may be a catalytically inactive form of a nuclease, such as a programmable nuclease described above. The protein may be a transcription activator-like effector (TALE). The protein may be complexed with a nucleic acid that guides the protein to an end of the target nucleic acid. For example, the protein may be a Cas endonuclease in a complex with one or more guide RNAs. In preferred embodiments, the protein is a Cas endonuclease, catalytically inactive Cas endonuclease, or homologs thereof.

In certain embodiments, the sample includes cfDNA from a subject. The sample is exposed to a first Cas endonuclease/guide RNA complex that binds to a target nucleic acid (e.g., a mutation of interest) in a sequence-specific fashion. In some embodiments, the complex binds to a mutation in a sequence-specific manner. A segment of the nucleic acid, i.e., the target nucleic acid, is protected by introducing the first Cas endonuclease/guide RNA complex and a second Cas endonuclease/guide RNA complex that also binds to the nucleic acid. In preferred embodiments of the method, the guide RNA comprises at least two guide RNA molecules that each complex with a Cas endonuclease and guide the Cas endonuclease to hybridize to one target nucleic acid, wherein the target nucleic acid includes a loci know to harbor a cancer-associated mutation.

In certain embodiments, the sample includes cfDNA from a subject. The sample is exposed to a first Cas endonuclease/guide RNA complex that binds to a target region of DNA (e.g., a region suspected to be hypermethylated) in a sequence-specific fashion. In some embodiments, the complex targets and binds to a one or more PAM sequences in a sequence specific manner that are adjacent the target region suspected to be hypermethylated. A segment of the DNA, i.e., the target region, is protected by introducing the first Cas endonuclease/guide RNA complex and a second Cas endonuclease/guide RNA complex that also binds to the DNA. In preferred embodiments of the method, the guide RNA comprises at least two guide RNA molecules that each complex with a Cas endonuclease and guide the Cas endonuclease to hybridize to a target region of DNA, wherein the target region includes a loci know to be hypermethylated.

Optionally, unprotected nucleic acid is digested. For example, one or more exonucleases may be introduced that promiscuously digest unbound, unprotected nucleic acid. Any suitable exonuclease may be used. Suitable exonucleases include, for example, Lambda exonuclease, RecJf, Exonuclease III, Exonuclease I, Exonuclease T, Exonuclease V, Exonuclease VII, T5 Exonuclease, and T7 Exonuclease, most of which are available from New England Biolabs (Ipswich, Mass.). While the exonucleases act, the target nucleic acid is protected by the bound complexes and survives the digestion step intact.

The described steps including the digestion by the exonuclease leave a reaction product that includes principally only the mutant segment of nucleic acid, as well as any spent reagents, Cas endonuclease complexes, exonuclease, nucleotide monophosphates, and pyrophosphate as may be present.

In certain embodiments, the exonuclease is deactivated. For example, exonuclease may be deactivated by following the manufacturer's instructions e.g., by heating to 90 degrees for a few minutes. After digestion, a positive selection step may be performed which may include, for example, amplification of the target nucleic acid by known methods or selection by an affinity assays.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a mutation. For example and without limitation, the feature may be an insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism. In other embodiments, the target region or feature of interest may be any site of epigenetic modification that is indicative of a disease, such as a nucleic acid modification, a histone modification, or chromatin remodeling. An example of a nucleic acid modification may be a modification to methylation status of DNA. As such, a feature of interest may be DNA hypermethylation or hypomethylation. In certain embodiments, the methods of the invention involve the detection of DNA hypermethylation. Histone modifications can also constitute an epigenetic modification indicative of disease. Examples of histone modifications include but are not limited to methylation, acetylation, deacetylation, ADP-ribosylation, ubiquitination or phosphorylation. Other examples of epigenetic elements indicative of disease include RNA interference (e.g., short interfering RNA, also known as siRNA) and prions.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target nucleic acid is from a bodily fluid sample from a human. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

The method optionally includes detecting the target nucleic acid (which may harbor the mutation). Any suitable technique may be used to detect the target nucleic acid. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid may indicate the presence of the mutation in the subject (i.e., a patient), and a report may be provided describing the mutation in the patient.

In an embodiment of the invention, detecting methylation in DNA of the target region is performed. Any suitable technique may be used to detect methylation in the target region of DNA. For example, detection may be performed using known hybridization assays, such as those that utilize antibodies, or other signal amplification techniques, such as an immunoassay, like an ELISA. The described methodology may be used to detect hypermethylation in the target region. In such cases, site-specific hypermethylation detection may be used to detect the presence of cancer.

In an embodiment of the invention, a sample may contain a mutant fragment of DNA, a wild-type fragment of DNA, or both. A locus of interest is identified where a mutation may be present proximal to, or within, a protospacer adjacent motif (PAM). When the wild-type fragment is present, it may contain a wild-type allele at a homologous location in the fragment, also proximal to, or within, a PAM. A guide RNA is introduced to the sample that has a targeting portion complementary to the portion of the mutant fragment that includes the mutation. When a Cas endonuclease is introduced, it will form a complex with the guide RNA and bind to the mutant fragment but not to the wild-type fragment. The first Cas endonuclease/guide RNA complex includes a guide RNA with a targeting region that binds to the mutation but that does not bind to other variants at a loci of the mutation. The described methodology may be used to target a mutation that is proximal to a PAM, or it may be used to target and detect a mutation in a PAM, e.g., a loss-of-PAM or gain-of-PAM mutation.

The described methodology may be used to target a mutation that is proximal to a PAM, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM includes NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over ctDNA at such frequency that the potentially detectable mutations are abundant enough as to be representative of mutations over the tumor DNA at large. In such cases, mutation-specific enrichment may be used to detect mutations from a tumor. Moreover, methods may be used to determine a number of mutations over the representative, targetable portion of tumor DNA. Since the targetable portion of the genome is representative of the tumor DNA overall, the number of mutations may be used to infer a mutational burden for the tumor.

In another embodiment of the invention, a sample may contain a mutant fragment of DNA, a wild-type fragment of DNA, or both. A locus of interest is identified where hypermethylation may be present adjacent to a protospacer adjacent motif (PAM). A guide RNA is introduced to the sample that has a targeting portion complementary to PAM sequences that are adjacent a target region suspected to be hypermethylated. When a Cas endonuclease is introduced, it will form a complex with the guide RNA and the complex will target and bind to the PAM sequences. The Cas endonuclease/guide RNA complex includes a guide RNA with a targeting region that binds to the PAM but that does not bind to other regions at a loci of the target region. The described methodology may be used to target and detect DNA methylation adjacent to a PAM. The described methodology may be used to target DNA methylation adjacent to a PAM, or it may be used to target a region and detect DNA hypermethylation adjacent a PAM. As such, the targetable portion of the DNA includes any twenty-three consecutive bases that terminate in GG. In such cases, enrichment may be used to detect methylated target regions of DNA known to be associated with cancer. Moreover, methods may be used to quantify DNA methylation levels of the target region of DNA. Since the targetable portion of the genome is representative of the tumor DNA overall, the hypermethylation levels may be used to infer a mutational burden for the tumor.

A feature of the method is that a specific mutation may be detected by a technique that includes detecting only the presence or absence of a fragment of DNA, and it need not be necessary to sequence DNA from a subject to describe mutations. Methods of the invention use protection at one or both ends of DNA segments. The gRNA selects for a known mutation on one end. A positive selection may be performed to positively select out the bound, target nucleic acid. If the gRNA does not find the mutation, no protection is provided and the molecule may be digested, e.g. in negative enrichment, and the remaining molecules are either counted or sequenced. Methods are well suited for the analysis of samples in which the target of interest is extremely rare, and particularly for the analysis of maternal plasma or serum (e.g., for fetal DNA) or a liquid biopsy (e.g., for ctDNA). Such methods of the invention are useful for detection of hypermethylation of target region of DNA known to be associated with cancer. The Cas endonuclease/gRNA complex targets and binds to PAM adjacent to the target region suspected to be hypermethylated.

Methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. It is noted that the Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection do not require PCR amplification and therefore significantly reduces cost and sequence bias associated with PCR amplification. Sample analysis can also be performed by a number of approaches, such as next generation sequencing (NGS), etc. However, many analytical platforms may require PCR amplification prior to analysis. Therefore, preferred embodiments of analysis of the reaction products include single molecule analysis that avoids the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

The target nucleic acid may be detected, sequenced, or counted. Where a plurality of fragments are present or expected, the fragment may be quantified, e.g., by qPCR.

The target nucleic acid may further be isolated or detected by any suitable method in order to separate the target segment from other nucleic acids in the sample. For example, the isolation or detection method may include separating the protein-bound target nucleic acid from some or all of the unbound nucleic acid. The isolation or detection method may include binding the protein-bound target nucleic acid to a particle. The particle may include magnetic or paramagnetic material. The isolation or detection method may include applying a magnetic field to the sample. The particle may include an agent that binds to a protein bound to an end of the target nucleic acid. The agent may an antibody or fragment thereof. The isolation or detection method may include chromatography. The isolation or detection method may include applying the sample to a column. The isolation or detection method may include separating the protein-bound target nucleic acid from some or all of the unbound nucleic acid by size exclusion, ion exchange, or adsorption. The isolation or detection method may include gel electrophoresis.

The target DNA may further be enriched or isolated by any suitable method in order to separate the target segment from other components in the sample. For example, the isolation or detection method may include separating the complex-bound target region DNA from some or all of the unbound DNA. In a preferred embodiment, the isolation method may include binding the complex-bound target region to a particle. The particle may include magnetic or paramagnetic material. The isolation or detection method may include applying a magnetic field to the sample. The particle may include an agent that binds to a protein bound to an end of the target DNA. The agent may an antibody or fragment thereof. The isolation or detection method may include chromatography. The isolation or detection method may include applying the sample to a column. The isolation or detection method may include separating the complex-bound target DNA from some or all of the unbound DNA by size exclusion, ion exchange, or adsorption. The enrichment or isolation method may include gel electrophoresis.

Embodiments of the invention may include detecting the target nucleic acid and optionally providing a report describing a mutation as present in the patient. In some embodiments, the target nucleic acid is a target region of DNA suspected to be hypermethylated. The mutation-containing fragments may be detected by a suitable assay, such as sequencing, gel electrophoresis, a probe-based assay. The detection of the isolated segment of the target nucleic acid may be done by sequencing. The detection of the methylation of the isolated target r may be done by any known signal amplification technique in the art. The digestion or the isolation provides a reaction product that includes principally only the target nucleic acid, as well as any spent reagents, Cas endonuclease complexes, exonuclease (e.g. when negative enrichment is performed), nucleotide monophosphates, or pyrophosphate as may be present. The reaction product may be provided as an aliquot (e.g., in a micro centrifuge tube such as that sold under the trademark EPPENDORF by Eppendorf North America (Hauppauge, N.Y.) or glass cuvette). The reaction product aliquot may be disposed on a substrate. For example, the reaction product may be pipetted onto a glass slide and subsequently combed or dried to extend the fragment across the glass slide. The reaction product may optionally be amplified. Optionally, adaptors are ligated to ends of the reaction product, which adaptors may contain primer sites or sequencing adaptors. The presence of the segment in the reaction product aliquot may then be detected using an instrument.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the nucleic acid may include identifying a mutation in the nucleic acid. In other embodiments, detecting the nucleic acid may include detecting methylation at the Identifying the mutation may include sequencing the nucleic acid (e.g., on a next-generation sequencing instrument), allele-specific amplification, and hybridizing a probe to the nucleic acid. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

One method for detection of protein-bound nucleic acids is immunomagnetic separation. Magnetic or paramagnetic particles are coated with an antibody that binds the protein bound to the segment, and a magnetic field is applied to separate particle-bound segment from other nucleic acids. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like. Preferably, the particles are coated with biotin-binding protein, such as Streptavidin. In other embodiments, the magnetic separation of the complex-bound regions provides for enrichment and isolation of the target region. The target region may be suspected to be hypermethylated.

Chromatographic methods may be used for detection. In such methods, the bodily fluid sample is applied to a column, and the target nucleic acid is separated from other nucleic acids based on a difference in the properties of the target nucleic acid and the other nucleic acids. Size exclusion chromatography is useful for separating molecules based on differences in size and thus is useful when the segment is larger than other nucleic acids, for example the residual nucleic acids left from a digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the target nucleic acid impart a differential charge as compared to other nucleic acids. Methods of ion exchange chromatography are known in the art and described in, for example, Small, Hamish (1989). Ion chromatography. New York: Plenum Press. ISBN 0-306-43290-0; Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the target nucleic acid is larger than other nucleic acids, for example the residual nucleic acids left from a digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Another method for detection is gel electrophoresis. Gel electrophoresis allows separation of molecules based on differences in their sizes and is thus useful when the target nucleic acid is larger than other nucleic acids, for example the residual nucleic acids left from a digestion step. Methods of gel electrophoresis are known in the art and described in, for example, Tom Maniatis; E. F. Fritsch; Joseph Sambrook. “Chapter 5, protocol 1”. Molecular Cloning—A Laboratory Manual. 1 (3rd ed.). p. 5.2-5.3. ISBN 978-0879691363; and Ninfa, Alexander J.; Ballou, David P.; Benore, Marilee (2009). fundamental laboratory approaches for biochemistry and biotechnology. Hoboken, N.J.: Wiley. p. 161. ISBN 0470087668, the contents of which are incorporated herein by reference.

Methods of the invention involve signal amplification techniques, such as antibody-based detection and analysis. In order to detect methylation of a target region of DNA the use of signal amplification techniques are employed. For example, antibody-based detection methods are generally based on the transformation of a specific biomolecular interaction between antigen and antibody into a macroscopically detectable signal or change in the physical properties of the media. See e.g., Sveshnikov, Peter; “The Potential of Different Biotechnology Methods in BTW Agent Detection: Antibody Based Methods” The Role of Biotechnology in Countering BTW Agents; Vol. 34 of the series NATO Science Series, pp. 69-77 (2001), incorporated herein by reference.

Particular antibody detection methods are known in the art. Proteins can be detected and quantified through epitopes recognized by polyclonal and/or monoclonal antibodies used in methods such as enzyme-linked immunoabsorbent assay (ELISA), immunoblot assays, flow cytometric assays, radioimmuno assays, immunocytochemical assays, Western blot assays, an immunofluorescent assays, chemiluminescent assays, flow cytometry and fluorescence-activated cell sorting (FACS), immunoprecipitation, enzyme linked immunospot (ELISPOT), and other polypeptide detection strategies. Proteins may also be detected by mass spectrometry assays (potentially coupled to immunoaffinity assays) including matrix-assisted laser desorption/ionization time-of-flight (MALDI-TOF) mass mapping and liquid chromatography/quadrupole time-of-flight electro spray ionization tandem mass spectrometry (LC/Q-TOF-ESI-MS/MS). Additionally, methods of the disclosed invention may include tagging of proteins separated by two-dimensional polyacrylamide gel electrophoresis (2D-PAGE), (Kiernan et al, Anal Biochem 301, 49-56 (2002); Poutanen et al, Mass Spectrom 15, 1685-1692 (2001) the content of each of which is incorporated by reference herein in its entirety) or any other method of detecting protein. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In some embodiments, immunohistochemistry methods may be used for detecting the presence of DNA hypermethylation of a target region. In these methods, antibodies (monoclonal or polyclonal) specific for each target region are used to detect hypermethylation. Immunohistochemistry protocols and kits are well known in the art and are commercially available.

Certain preferred embodiments include obtaining a blood, plasma, or serum sample from a patient. The blood, plasma, or serum may include cfDNA and thus also include ctDNA among the cfDNA. Specific sequences of the ctDNA are isolated or enriched and analyzed or detected to detect or report genetic information from the subject, such as a presence or count of certain tumor mutations. Methods of the invention include introduce Cas endonucleases (or catalytically inactive homologs thereof such as dCas9) directly into serum or plasma. The Cas endonucleases are complexed with guide RNAs that include targeting portions specific for a target nucleic acid. In the plasma or serum, the complexes bind to ends of the target and protect it. Exonuclease may be introduced to digest unbound nucleic acid into monomers and fragments too small for further meaningful detection, sequencing, or amplification.

Embodiments of the invention provide for treatment of a sample. For example, a blood sample may be obtained from a patient. The sample may be collected in any suitable blood collection tube such as the collection tube sold under the trademark VACUTAINER by BD (Franklin Lakes, N.J.). In certain embodiments, the collection tube comprises an EDTA collection tube, and Na-EDTA collection tube or the collection tube sold under the trademark CELL-FREE DNA BCT by Streck, Inc. (La Vista, Nebr.), sometimes referred to in the art as a Streck tube. Use of a Streck tube stabilizes nucleated blood cells and prevents the release of genomic DNA into the sample. This facilitates the collection of sample that includes cell-free DNA.

The sample may be centrifuged to generate a sample that includes a pellet of blood cells and a supernatant, which contains serum or plasma. Serum is the liquid supernatant of whole blood that is collected after the blood is allowed to clot and centrifuged. Plasma is produced when the process includes an anticoagulant. To collect serum, blood is collected in tubes. After collection, the blood is allowed to clot by leaving it undisturbed at room temperature (about 15-30 minutes). The clot is removed by centrifuging, e.g., at 1,000-2,000×g for 10 minutes in a refrigerated centrifuge. The resulting supernatant is designated serum and may be transferred to a clean polypropylene tube using a Pasteur pipette. For plasma, blood is collected into commercially available anticoagulant-treated tubes e.g., EDTA-treated (lavender tops), citrate-treated (light blue tops), or heparinized tubes (green tops), followed by centrifugation to collect the supernatant. The supernatant is preferably transferred to a fresh tube, away from the pellet, which may be discarded. Particularly where the collection tube included an anticoagulant, the transfer should give a good separation of the plasma from the whole blood cells. After transfer, the sample includes plasma or serum, which includes cfDNA.

In an exemplary embodiment, serum or plasma is transferred from a centrifuge tube to a new tube, complexes comprising Cas9 and guide RNA are added, and the mixture is incubated. For example, amplification or an affinity assay may be performed to positively select out the bound, target nucleic acid. In another embodiment, exonuclease may be introduced to digest unbound, non-target DNA, and then the exonuclease may be deactivated (e.g., by heat). A positive selection may then follow (e.g., amplification or an affinity assay) to positively select out the bound, target nucleic acid.

In another exemplary embodiment, plasma or serum is removed from the centrifuge tube (the supernatant) and transferred into a new tube. Appropriate buffers/reagents are added to modify a chemical environment to promote binding of Cas endonuclease to the target nucleic acid. For example, pH can be adjusted, as may temperature, salinity, or co-factors present. The Cas complexes are added and allowed to incubate. For example, amplification or an affinity may be performed to positively select out the bound, target nucleic acid. An exonuclease may optionally be added, which ablates all free, non-target nucleic acid. The target may be positively selected such as by amplification or an affinity assay after exonuclease digestion of the non-target nucleic acid.

Methods may include detection or isolation of circulating tumor cells (CTCs) from a blood sample. Cytometric approaches use immunostaining profiles to identify CTCs. CTC methods may employ an enrichment step to optimize the probability of rare cell detection, achievable through immune-magnetic separation, centrifugation, or filtration. Cytometric CTC technology includes the CTC analysis platform sold under the trademark CELLSEARCH by Veridex LLC (Huntingdon Valley, Pa.). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

Certain embodiments of the invention may provide a kit. The kit preferably includes reagents and materials useful for performing methods of the invention. For example, the kit may include one or more guide RNA that, taken in pairs, are designed to flank cancer-associated mutations. The kit may include one or more guide RNAs that are mutation specific and only hybridize to target that includes a mutation. The kit may include a Cas endonuclease or a nucleic acid encoding a Cas endonuclease such as a plasmid. The kit may optionally include exonuclease. The kit may include reagents for adjusting conditions such as pH, salinity, co-factors, etc., to promote binding or activity of Cas endonuclease (including to promote binding of catalytically inactive Cas endonuclease, which may be included as the Cas endonuclease) in the bodily fluid sample, such as plasma or serum. The kit may further include instructional materials for performing methods of the invention, and components of the kit may be packaged in a box suitable for shipping or storage. Preferably, the kit contains one or more collection tubes, such as a blood collection tube. In other embodiments, the kit may include one or more guide RNA that, taken in pairs, are designed to flank cancer-associated methylation sites. The kit may include one or more guide RNAs that are target specific and only hybridize to PAM sequences adjacent to the target region that includes a methylation site suspected to be hypermethylated.

The Cas endonuclease/guide RNA complexes can be designed to bind to mutations of clinical significance, such as a mutation specific to a tumor. In other embodiments, the complexes can be designed to target and bind to one or more target regions of DNA suspected to be hypermethylated, where such hypermethylation is specific to a tumor. When a mutation is thus detected, a report may be provided to, for example, describe the mutation in a patient or a subject. Thus, certain embodiments may comprise providing a report. The report preferably includes a description of the mutation in the subject (e.g., a patient). The method for detecting rare nucleic acid may be used in conjunction with a method of describing mutations (e.g., as described herein). Either or both detection processes may be performed over any number of loci in a patient's genome or preferably in a patient's tumor DNA. As such, the report may include a description of a plurality of structural alterations, mutations, or both in the patient's genome or tumor DNA. As such, the report may give a description of a mutational landscape of a tumor. In other embodiments, the report may provide a description of the DNA methylation in the target region of the subject.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor. Furthermore, knowledge of DNA methylation of a target region may also be used similarly.

Methods of the invention thus may be used to detect and report clinically actionable information about a patient or a tumor in a patient. For example, the method may be used to provide a report describing the presence of the genomic alteration in a genome of a subject. In other embodiments, the method may be used to provide a report describing the DNA methylation of a target region known to be associated with cancer of a subject. Additionally, protecting a segment of DNA, and optionally digesting unprotected DNA, provides a method for isolation or enrichment of DNA fragments, i.e., the protected segment. It may be found that the described enrichment techniques are well-suited to the isolation/enrichment of arbitrarily long DNA fragments, e.g., thousands to tens of thousands of bases in length or longer.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest.

The methods described herein provide the ability to assay for methylation and other epigenetic changes that are indicative of disease. Methods of the invention are conducive to high-throughput testing, and may be performed, for example, in droplets on a microfluidic device, to rapidly assay a large number of aliquots from a sample for one or any number of genomic structural alterations. Furthermore, using the methods described herein, a biological sample can be assayed for hypermethylation or other epigenetic changes in target regions using a technique that does not require bisulfite treatment of DNA to detect methylation.

### Example

The cutting efficiency of amplicons by Cas9 in plasma is shown by experiment. Results from the experiment indicated that Cas proteins bind to expected cognate targets under guide RNA guidance in plasma or serum. In particular, Cas9 was tested for cutting activity in plasma in an experimental protocol.

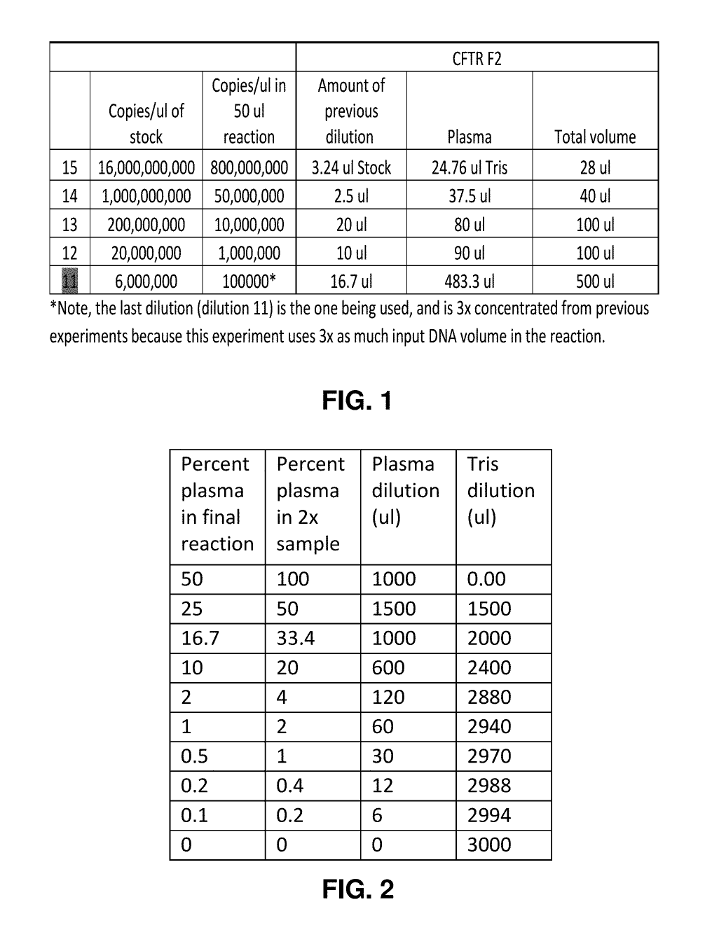

Plasma samples were placed in Streck tubes and in standard tubes. The experiments used an 800 bp amplicon from the cystic fibrosis transmembrane receptor gene. Dilutions were made of CFTR F2 800 bp into plasma with 5 million copies per reaction total (FIG. 1). The percent plasma in reaction after dilution was 50%, 25%, 16.7%, 10%, 2%, 1%, 0.5%, 0.2%, 0.1%, and 0% (FIG. 2).

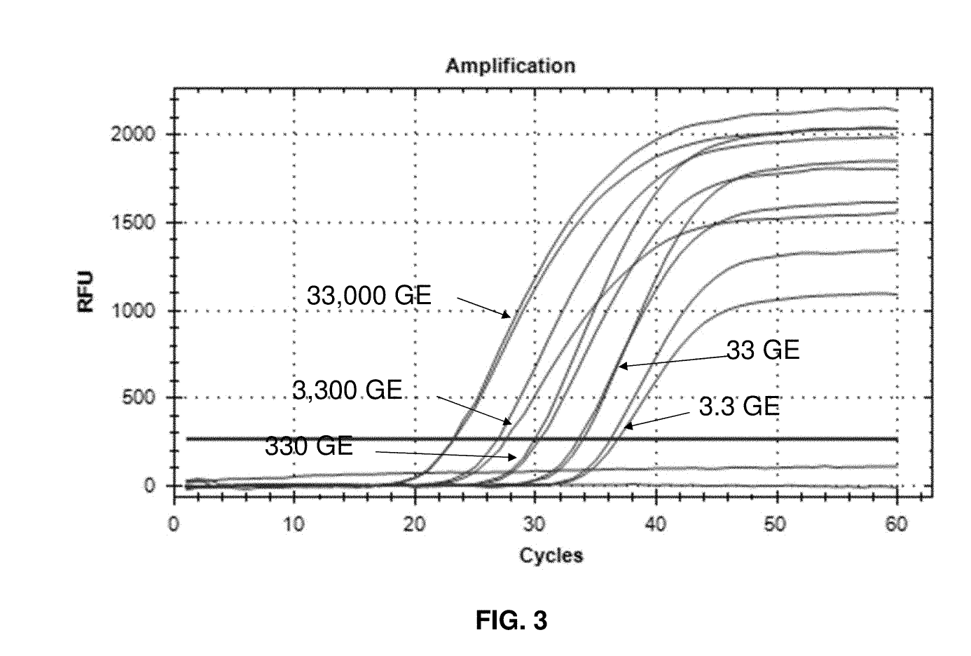

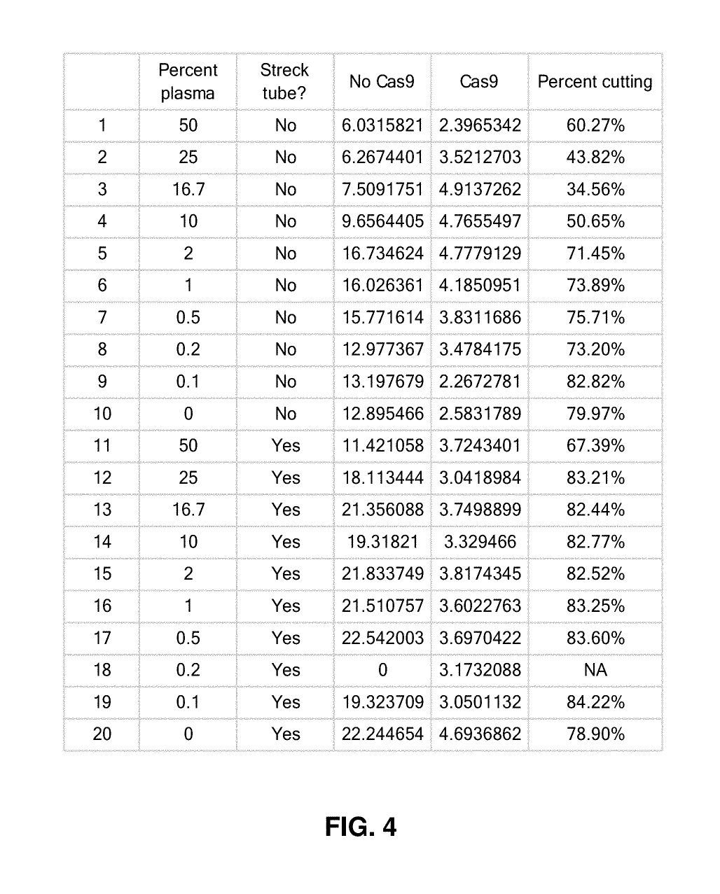

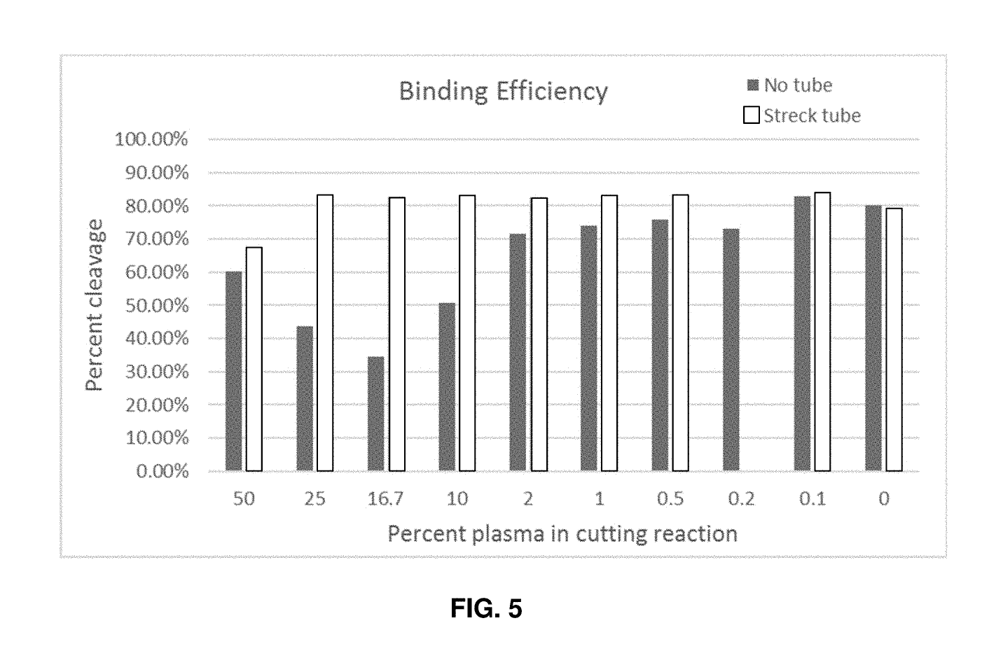

Cas9 with guide RNA was added and allowed to cut. qPCR was then used to probe across the cut site. For qPCR, samples were diluted 1/100, and then 5 ul were used per 20 ul reaction. The qPCR results were analyzed from amplifying, post-cutting, from dilutions (FIGS. 3 and 4). The qPCR results indicated cleavage as a function of plasma amount (FIG. 5). For example, every replicate in a Streck tube demonstrated greater than 60% cutting efficiency by Cas9 in the CFTR amplicon. Cas9 exhibited detectable cutting, even in standard, non-Streck tubes.

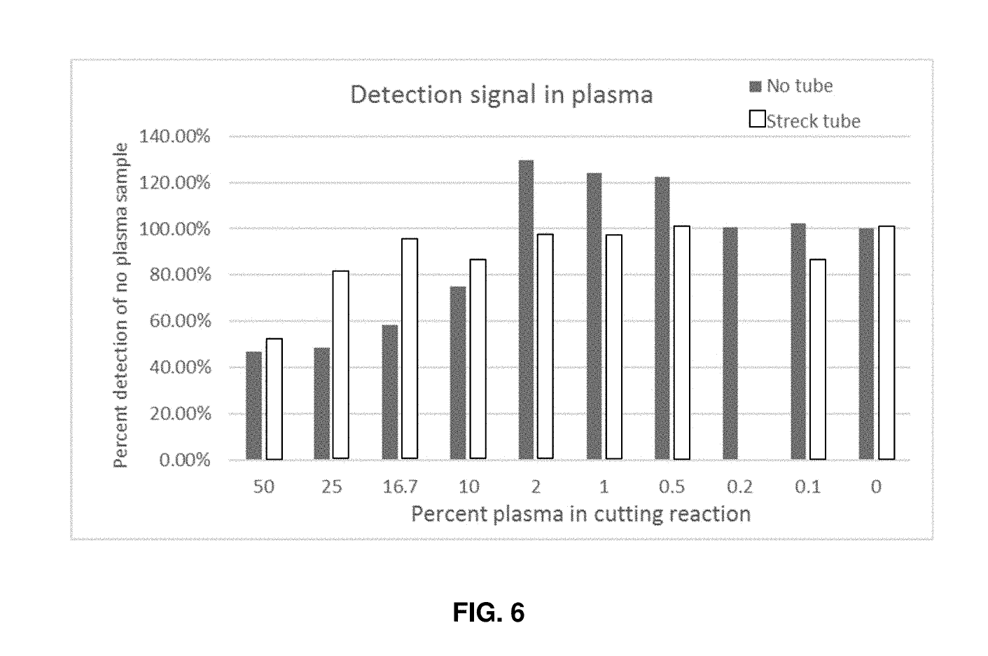

The results also indicated a relationship between the qPCR signal and percent plasma (FIG. 6). For example, the data show Cas9 exhibits detectable cutting in Na-EDTA plasma. For the reactions performed in straight plasma, cutting efficiency in 2% plasma or lower resembled no plasma cutting efficiency (82.82% for in plasma compared to 79.97% in no plasma). For the reactions performed in plasma incubated in a Streck tube, the cutting efficiency in 25% plasma or lower resembled no-plasma cutting efficiency (83.14% compared to 78.90%). Further, there was 60-67% cutting for the 50% plasma samples. In 50% plasma, CRISPR/Cas9 complexes retained 75% activity. Results of the data show that Cas endonuclease and homologs thereof bind to target DNA under guidance of guide RNA in plasma.

In another example, sequence specific Cas9/gRNA complexes were added directly to a biological sample, a plasma sample. Here, a complex of Cas9-guide RNA and a particle, in this example, a magnetic bead coated with streptavidin, is directly inserted into the plasma sample. That is, the magnetic bead bound Cas proteins bind to expected targets under guide RNA guidance in plasma (or serum) and cut and protect the target region to achieve enrichment. In this example the target region is suspected to be hypermethylated and the complexes were complementary to both sides the target region of DNA. The complexes therefore targeted and bound to PAM sequences that were adjacent to the target region suspected to be hypermethylated.

The particle-bound complexes were then isolated from the remaining sample using a separator, in this case, a magnetic field was used to separate out the particle-bound complexes. Once target specific positive enrichment was completed, antibodies that bind only to methylated DNA, and not un-methylated DNA were added to the enriched sample. An ELISA was then performed to detect the signal produced by the binding of the methylation specific antibodies to the methylated target region of DNA. Results from the experiment indicated that Cas proteins bind to expected target regions that are suspected to be hypermethylated under guide RNA guidance in a liquid biopsy, such as plasma or serum, allowing for the use of binding assays, such as an ELISA to detect the presence of methylation at the specific target region. Such an ability to detect the methylation status of known cancer-associated regions, allows for the early detection of cancer and tumor growth.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for detecting methylation in DNA, the method comprising:
exposing a biological sample to a Cas endonuclease/guide RNA complex that binds to one or more target regions of DNA suspected to contain one or more epigenetic modifications;
enriching the sample by isolating said target regions; and
detecting said target regions.

2. The method of claim 1, wherein the epigenetic modification is hypermethylation.

3. The method of claim 1, wherein the detection step is carried out by one or more means selected from the group comprising ligand binding assay, immunoassay, western blot analysis, hybridization, amplification, chromatography, and fluorescence detection.

4. The method of claim 1, wherein the detection step comprises using an antibody that binds to methylated DNA in the target region and performing an immunoassay to detect said antibody.

5. The method of claim 4, wherein the detection of the antibody is indicative of methylation in the target region.

6. The method of claim 5, wherein detection of hypermethylation in the target region is indicative of a tumor in the sample.

7. The method of claim 5, further comprising:
quantifying relative amounts of methylation of the target region.

8. The method of claim 1, wherein the Cas endonuclease is a catalytically inactive homolog thereof.

9. The method of claim 1, wherein the enriching step further comprises introducing an exonuclease to the sample to digest unbound nucleic acid.

10. The method of claim 1, wherein the enriching step comprises connecting the complex-bound target region to a particle or column and removing other components of the sample.

11. The method of claim 10, wherein the particle comprises an agent that binds to at least one Cas endonuclease to form a particle-bound segment.

12. The method of claim 10, wherein the particle comprises magnetic or paramagnetic material and the enriching step further comprises applying a magnetic field to separate the particle-bound segment from the other components.

13. The method of claim 1, wherein the enriching step comprises applying the sample to a column.

14. The method of claim 1, wherein the complex-bound target region is separated from unbound nucleic acid in the sample by size exclusion, ion exchange, or adsorption.

15. The method of claim 1, wherein the enriching step comprises gel electrophoresis.

16. The method of claim 1, wherein the sample comprises bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, or vomit.

17. The method of claim 1, wherein the target region comprises cDNA, cfDNA, or ctDNA.

18. The method of claim 17, wherein the target region is present at no more than about 0.01% of cell-free DNA in the sample.

19. The method of claim 18, wherein target region comprises an oncogene.

20. The method of claim 19, wherein the oncogene is a tumor suppressor gene.

21. The method of claim 1, wherein the complexes are targeted to PAM sequences that are near a target region suspected to be hypermethylated.
